(19) United States
(12) Patent Application Publication
(10) Pub. No.: US 2005/0043716 A1
(43) Pub. Date: Feb. 24, 2005

(54) Implantable prosthesis and method of use

(75) Inventor: Michael Lewis Frimer, Richmond (CA)

Correspondence Address: Jason M. Honeyman;Wolf, Greenfield & Sacks, P.C., 600 Atlantic Avenue, Boston, MA, 02210, US

(73) Assignee: C.R. Bard, Inc., Murray Hill, NJ (US)

(21) Appl. No.: 10/755,422

(22) Filed: Jan. 12, 2004

Related U.S. Application Data
(60) Provisional application No. 60/446,257, filed on Feb. 11, 2003.

(51) Int. Cl. A61B 17/00

(52) U.S. Cl. 606/1

(57) ABSTRACT

An implantable prosthesis and a system and method for repairing, or resisting the formation of, a hernia at an opening or stoma through an anatomical structure such as the abdominal wall. The implantable prosthesis includes a body portion and an opening therethrough, that is adapted to receive an element, such as a section of bowel, that is being externalized through the anatomical structure. The system includes the implantable prosthesis and a cannula having an outer dimension sized to fit within the opening in said implantable prosthesis, and an inner dimension sized to pass through the element. The implantable prosthesis may include a reinforcement member, and it may also include flaps that extend into the opening of the implantable prosthesis. The method includes inserting the cannula into and beyond the opening or stoma, and locating the opening of the prosthesis about the end of the cannula. A portion of the element to be externalized, such as a section of bowel in an enterostomy, is drawn through the end of the cannula such that the bowel section does not contact the implantable prosthesis. The cannula is then removed.

### RELATED APPLICATIONS

This application claims the benefit under 35 U.S.C. §119(e) to U.S. Provisional Application Ser. No. 60/446,257, entitled “Method and Apparatus for the Standardized Creation of Permanent Enteral Stomas and Prevention of Parastomal Hernias,” filed on Jan. 12, 2003, which is herein incorporated by reference in its entirety.

### FIELD OF INVENTION

The present invention relates to an implantable prosthesis and a method for repairing, or resisting the formation of, a hernia at an opening or stoma through an anatomical structure.

### DISCUSSION OF RELATED ART

In certain surgical procedures, it is known to form an opening in an anatomical wall (i.e., a “stoma”), to allow passage of a structure therethrough. In an enterostomy (e.g., colostomy, ileostomy, and urostomy), a portion of the bowel is relocated through an opening formed in the abdominal wall. Permanent enterosomies may be performed, for example, in the treatment of rectal cancer, inflammatory bowel disease, and urinary bladder cancer. Representative is a colostomy, where the rectum is removed. The remaining end of the bowel is rerouted through a stoma formed in the abdominal wall into a bag or other colostomy appliance attached to the skin of the patient, where bowel wastes and/or urine may be collected.

The surgical formation of a stoma creates a potential for weakness in the abdominal wall at and/or near the opening. Over time, stretching of the abdominal wall due to strain such as coughing, sneezing, standing up and sitting down, may further weaken the abdominal wall surrounding the stoma, potentially allowing intra-abdominal contents to protrude into or next to the stoma, which present as a bulge at or near the stoma. Such ruptures or defects are known as parastomal hernias.

It is an object of the present invention to provide an implantable prosthesis for the repair and prevention of a hernia around an opening or a stoma. It is a further object of the present invention to provide a method for repairing and preventing a hernia around an opening or a stoma.

### SUMMARY OF INVENTION

In one embodiment of the invention, an implantable prosthesis is provided for repairing, or resisting the formation of, a hernia at an opening or stoma formed in an anatomical structure, where an anatomical or prosthetic element extends through the opening or stoma. The implantable prosthesis includes a body portion for placement against the anatomical structure that at least partially surrounds the stoma, and an opening therethrough that is adapted to receive a portion of the element that is passed through the stoma. The implantable prosthesis is effective for at least the repair, or resistance to formation of, a hernia in the anatomical structure at or near the opening or stoma.

In another embodiment of the present invention, a system is provided for repairing, or resisting the formation of, a hernia at or near a stoma formed in the abdominal wall, wherein a portion of bowel extends through the stoma. The system includes an implantable prosthesis having a body. portion for augmenting or repairing the weakened abdominal wall at or near the stoma, and an opening therethrough that is adapted to receive the portion of the bowel that extends through the stoma. The implantable prosthesis is effective for at least one of the repair, or resistance to formation, of a hernia in the abdominal wall at or near the stoma. The system further includes a cannula, separate from the implantable prosthesis, having an outer dimension that is sized to fit within the opening in the implantable prosthesis, and an inner dimension that is sized to pass the portion of bowel therethrough, the cannula being removable from, that is not permanently connected to, the opening in the implantable prosthesis. The system may further include a trocar stylet for forming the opening through the abdominal wall, and may also include one or more sizing cylinders for aiding in selection of the appropriate trocar stylet for the involved procedure. The implantable prosthesis and cannula may be provided in a kit, along with one or more of the following: at least one sizing cylinder, at least one trocar stylet, and instructions for using any of the components provided in the kit to perform an externalization procedure, such as an enterostomy, or the repair of a parastomal hernia, preferably to avoid contamination of the implantable prosthesis during relocation of the anatomical structure being externalized, such as a section of bowel in an enterostomy.

In another embodiment of the present invention, a method is provided of repairing, or reducing the incidence of, a hernia at or near a stoma formed in an abdominal wall through which a portion of bowel extends. An implantable prosthesis is provided with an opening therethrough that is adapted to receive the bowel portion. A shield, such as a cannula, is provided having an outer dimension that is sized to fit within the opening of the implantable prosthesis and an inner dimension that is sized to pass the bowel portion therethrough. The cannula is located in the stoma, and at least one of the cannula and the implantable prosthesis are positioned so that an end of the cannula extends through the opening in the implantable prosthesis. The bowel portion is passed through the cannula, limiting, and preferably avoiding, contact with the implantable prosthesis and/or contamination of the implantable prosthesis by any bacteria carried by the bowel portion. The cannula is then removed.

### BRIEF DESCRIPTION OF DRAWINGS

The foregoing and other objects and advantages of the invention will be appreciated more fully from the following drawings, wherein like reference characters designate like features, in which:

FIG. 1 is a top plan view of an implantable prosthesis in accordance with one illustrative embodiment of the present invention;

FIG. 2 is a cross-sectional view of the implantable prosthesis of FIG. 1 taken along line A-A;

FIG. 3 is a cross-sectional view of the implantable prosthesis of FIG. 1 similar to FIG. 2 except that the implantable prosthesis is shown implanted in the abdominal wall with a portion of the bowel positioned therethrough;

FIG. 4 is an assembly view of a trocar stylet and cannula assembly;

FIG. 5 is a cross-sectional view of the trocar stylet and cannula assembly inserted into the abdominal wall;

FIG. 6 is a cross-sectional view of the removal of the trocar stylet from the cannula within the abdominal wall;

FIG. 7 is a cross-sectional view of the implantable prosthesis positioned about the cannula;

FIG. 8 is a cross-sectional view of the retraction of the bowel from the abdominal cavity up to the skin surface;

FIG. 9 is a cross-sectional view of the removal of the cannula;

FIG. 10 is a cross-sectional view of the bowel and implantable prosthesis sutured in place;

FIG. 11 is a top plan view of the implantable prosthesis positioned about the bowel;

FIG. 12 is a cross-sectional view of the implantable prosthesis of FIG. 11 taken along line B-B;

FIG. 13 is a side view of a plurality of sizing cylinders;

FIG. 14 is a cross-sectional view of the abdominal wall thickness measurement;

FIG. 15 is a cross-sectional view of a sizer used to measure the bowel for the appropriate sized cannula;

FIG. 16 is a cross-sectional view of a sizer used to size the bowel to measure the size of the required buttonhole; and

FIG. 17 is a cross-sectional view of the sizer of FIGS. 15-16 used as a template for marking the buttonhole and the medial and lateral extension cuts.

### DETAILED DESCRIPTION

Although the implantable prosthesis and method of use are described, principally, in connection with the repair, or resistance to formation, of a parastomal hernia associated with an ilesotomy, colostomy, urostomy, and other procedures involving externalization of a section of the bowel through a stoma in an abdominal wall, the invention is not so limited, and the inventive prosthetic device, and the inventive technique of relocating a body structure through a prosthetic device, preferably to limit or avoid contact therebetween so as to reduce the occurrence of potential contamination of the implant, has other applications as should be apparent to one of skill in the art. For purposes of this specification and the claims, “externalization” means the relocation of an anatomical structure, such as the bowel, or a prosthetic device or other structure, from the interior side of an anatomical wall to the exterior of the anatomical wall, and “stoma” means an opening in an anatomical wall that is naturally or non-naturally formed (e.g., the result of a surgical procedure).

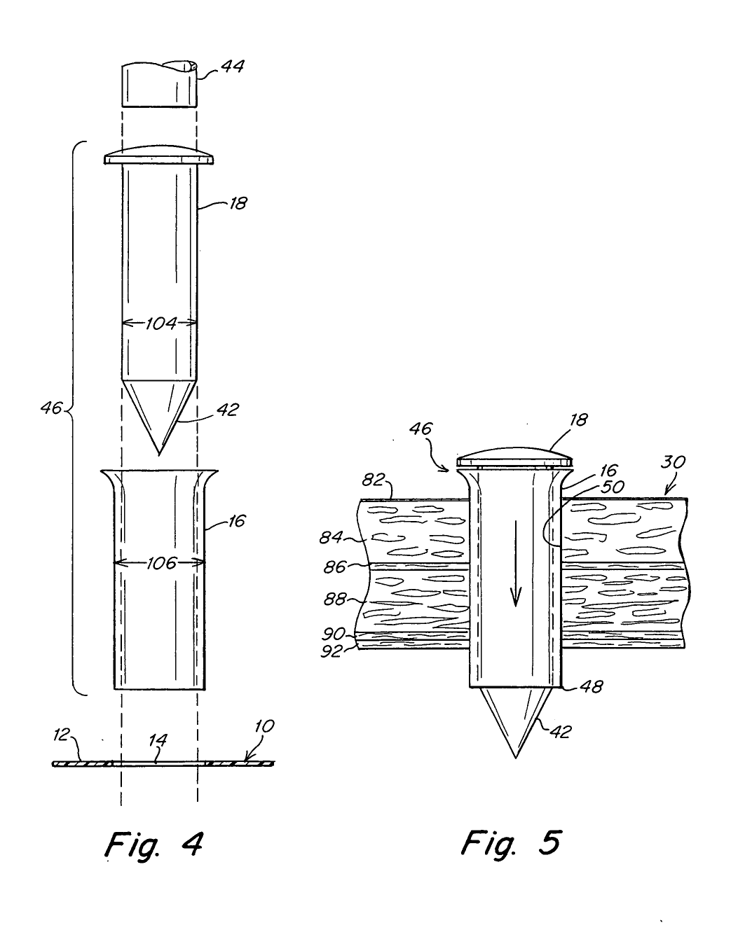

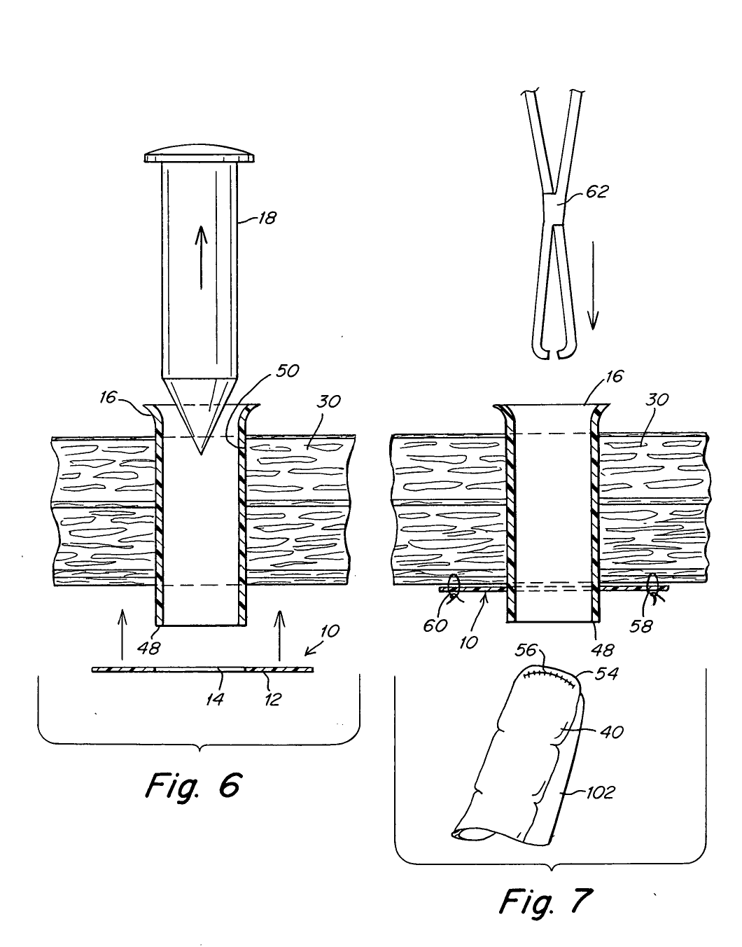

Turning to FIGS. 1-3, an implantable prosthesis 10 for repairing, or resisting the formation of, a hernia, particularly a parastomal hernia, is illustrated and includes a body portion 12 having an opening 14 for receiving a section of bowel 40 or other body structure that is intended to be externalized. The implant may be positioned so that the opening 14 is registered with a stoma in the abdominal wall, providing a pathway for the bowel through the implant and the abdominal wall to the skin surface, where the externalized bud may be positioned as required for the particular procedure. As disclosed below, the implantable prosthesis may be used in conjunction with a shield, such as cannula 16 (FIG. 4), to facilitate passage of the bowel from inside of the abdominal cavity to the outside, while reducing the incidence of bacterial contamination of the implant during externalization.

The opening 14 preferably is the same size or slightly smaller than the entry to the stoma formed through the abdominal wall, so that gaps between the body portion and the entrance to the stoma are avoided which might otherwise be vulnerable to herniation. The body portion may be formed of a prosthetic repair material having properties (e.g., strength, resistance to elongation, stiffness) for an effective repair, or resistance to formation, of a parastomal hernia. The body portion may be partially or wholly tissue infiltratable, or may be impervious to tissue ingrowth. In certain embodiments where the implant is not amenable to tissue infiltration, the device may become integrated with tissue that grows around the body portion of the prosthesis. The implant may, in an unstressed or natural state, such as prior to implantation, have a generally flat or planar shape, or may be arranged with a concave and/or convex shape on one or more surfaces, or may include a more complex three dimensional shape. The prosthetic device preferably is flexible, facilitating handleability and placement of the implant, as well as comfort of the patient post implantation. In certain embodiments, the implant may be collapsible, such as by folding, rolling, or otherwise. The flexibility of the implant may be influenced by many factors including, but not limited to, the materials from which the implant is constructed, the provision of any shape influencing members, treatments applied to the material of the implant, and the amount of stitching or other attachment features in the body of the implant.

In the particular embodiment shown in FIGS. 1-3, the implantable prosthesis includes a first layer 20 and a second layer 22. The first layer may be tissue infiltratable while the second layer may be a barrier to tissue ingrowth. In other embodiments, the orientation of the tissue ingrowth and barrier layers may be reversed. In still further embodiments, the implantable prosthesis may include one or more tissue infiltratable portions and barrier portions in the same layer. The selection, location, and number of tissue infiltratable and/or barrier portions of the implant may be dependent upon the ultimate application as should be apparent to one of skill in the art, and the number of layers of a particular prosthetic material may vary in different portions of the implant. In the colostomy illustrated in FIG. 3, a tissue infiltratable surface is positioned against the abdominal wall while a barrier surface faces the abdominal viscera. As observed earlier, the body portion may include exclusively a tissue infiltratable portion, exclusively a barrier portion, or a combination or one or more tissue infiltratable and barrier portions. In certain embodiments, the body portion defining the opening may constitute a tissue infiltratable portion only, a barrier portion only, or a combination of a tissue infiltratable portion and a barrier portion.

In one embodiment, the prosthetic device includes a sheet of biologically compatible, flexible, prosthetic repair fabric having a plurality of interstices or openings which allow tissue ingrowth, integrating the repair device to host tissue after implantation. A representative material is knitted polypropylene monofilament mesh, such as BARD MESH, available from C. R. Bard, Inc. When implanted, the polypropylene mesh promotes rapid tissue ingrowth into and/or around the mesh structure. Alternatively, other surgical materials which are suitable for tissue reinforcement in defect closure may be utilized including, without limitation, polytetrafluoroethylene (PTFE) mesh, PROLENE, SOFT TISSUE PATCH (microporous ePTFE), SURGIPRO, TRELEX, ATRIUM, MERSELENE, non-absorbable collagen, and polyester. Absorbable materials, including polyglactin (VICRYL), polyglycolic acid (DEXON), and absorbable collagen may also be employed. It is contemplated that the fabric may be formed from monofilament or multifilament yarns which may be woven, knitted, molded, or otherwise interengaged to form the tissue infiltratable component of the implant.

In an embodiment where the body portion is characterizable as non-tissue infiltratable, the barrier may be formed from a sheet of expanded polytetrafluoroethylene (ePTFE), such as GORE-TEX available from W. L. Gore & Associates, Inc., having a pore size (submicronal) that discourages tissue ingrowth and adhesion. A representative and non-limiting sampling of other suitable barrier materials includes silicone elastomer, such as SILASTIC Rx Medical Grade Sheeting (Platinum Cured) distributed by Dow Corning Corporation, TEFLON mesh, microporous polyproplyene sheeting (CELGARD), collagen, hyaluronic acid, carboxymethyl cellulose, and glycolic acid polymers. Autogenous, heterogeneous, and xenogeneic tissue also are contemplated including, for example, pericardium and small intestine submucosa. Absorbable materials, such as oxidized, regenerated cellulose (INTERCEED (TC7)) may be employed for some applications. The barrier can be a blend, mixture, or hydrogel of any of the materials to form a temporary or permanent barrier.

As observed above, and as described in connection with the device illustrated in FIGS. 1-3, the prosthesis may include both tissue infiltratable and barrier portions. In these embodiments, a combination of any of the representative materials identified above may be used, as well as other suitable materials, as should be apparent to one of skill in the art. Further, a tissue infiltratable material (such as BARD MESH), or a barrier material (such as ePTFE), may be treated or otherwise altered so that certain portions of a tissue infiltratable material become impervious to tissue ingrowth, and/or certain portions of a barrier material become susceptible to tissue ingrowth. For example, one or more portions of a tissue infiltratable fabric layer may be melted and resolidifed in non-porous form to render those portions resistant to tissue ingrowth. Other suitable techniques may include ultrasonic, induction, vibration, infrared/laser welding and the like. The barrier portions of the implant may also include the outer edge of the prosthetic device, as well as the inner edge circumscribing the opening. In certain embodiments, a segment of the tissue infiltratable portion adjacent the outer edge, and/or a segment of the tissue infiltratable portion that is adjacent the opening may be configured as a barrier to tissue ingrowth.

The implantable prosthesis may be reinforced at or near the opening to further reduce the likelihood of a parastomal defect. Where reinforced, the implant will exhibit at least one property (for example, but without limitation, strength, resistance to elongation, stiffness) effective for repairing, or reducing the incidence of formation of a parastomal hernia, that is superior to the same property of a body portion adjacent to the reinforced portion. For example, and without limitation of the invention, the implantable prosthesis may further include a reinforcement portion 24 as shown in FIGS. 1-3. The reinforcement portion 24 may be located at the opening 14, or it may be spaced away from the opening 14. The reinforcement portion 24 is not limited to a particular configuration, and for example may be ring shaped as illustrated and may extend completely, or only partially, around the opening 14. Further, the reinforcement portion may include discrete segments that are spaced from one another. The reinforcement portion may be formed of one or more materials included in the body portion of the prosthesis, such as by heating or otherwise treating the body portion to make a segment at or near the opening less susceptible to tearing, stretching, or other deformation that might lead to herniation.

The reinforcement portion may include a separate member 24 that is joined to the body portion. For example, and without restricting the scope of the reinforcement feature, a ring or other shape of an implantable material may be integrated with the body portion. Various arrangements for joining the reinforcement member to the body portion are contemplated, including fixing the ring to the body portion, such as by one or more of bonding, stitching, or fusing the components together. It also is contemplated that the reinforcement member may be fixed to more than one surface of the body portion, such as to both a top and bottom surface of the implant. It also is contemplated that a reinforcement member may be sandwiched between various layers of a multilayer body portion. The separate reinforcement member may be formed of the same material as is included in the body portion of the prosthesis or may consist of another biologically compatible and implantable material. In one embodiment, the body portion may comprise three layers, with a top layer formed of tissue infiltratable material, a bottom layer formed of a barrier material, and a third layer sandwiched between the top and bottom layers, that reinforces the body portion at or near the opening. In this embodiment, the reinforcement member may join the top and bottom layers together. In another embodiment, the reinforcement member is attached to an edge of the body portion. For example, and without limitation, such a reinforcement member may have a ring shape and include a U-shaped channel at its outer edge, defining a top rim that may be fixed to a top surface of the body portion, and a bottom rim that may be fixed to a bottom surface of the body portion. Further, an inner wall of the channel may be fixed to the inner edge of the body portion.

The implantable prosthesis 10 may also include, as shown in FIGS. 1-3, a shape influencing member 26 that may help maintain a desired shape of some portion or all of the prosthesis 10. The shape influencing members may be in the form of thin strips or filaments of metal, polymer, and the like, that may be engaged to, or otherwise in contact with, the implant and naturally or upon application of a force (e.g., heat) cause the prosthesis to form a predetermined shape. For example, where it is desired that the implantable prosthesis have a substantially flat configuration, the shape influencing member would act to assist in returning the implant, if it becomes folded or otherwise altered from its desired orientation, to a planar shape. In the embodiment illustrated in FIGS. 1-3, the shape influencing member is located near the outer portion of the implant to help ensure that the edges of the prosthesis stay or revert to the desired shape. The shape influencing member may have a variety of configurations that will help induce the implant to maintain or return to a desired shaped. Representative configurations include a ring as shown, other annular arrangements, a criss-cross, radially extending segments, and other designs as should be apparent to one of skill in the art. The shape influencing member may also serve as a site for anchoring sutures or other fixation devices between the implantable prosthesis and the surgical site. In certain embodiments, a retention member that does not influence the shape of the prosthesis, but otherwise is similarly configured to the shape influencing member just described, may be provided.

Certain embodiments of the implantable prosthesis 10 may include at least one flap 28 or tab that cooperates with the externalized bowel or other body structure that passes through the implant. For example, it may be desirable to attach the bowel to the implant to help secure the position of the bowel 40 with respect to the skin, preventing return (e.g., prolapse) of the externalized section of bowel 40 into the abdominal cavity. With that objective, one or more flaps or tabs may be tissue infiltratable. In other approaches, it may be preferred to avoid integration between the bowel and the implant, and the one or more flaps or tabs may be in the form of a barrier. In either case, the one or more flaps may radially extend into the opening 14 and unfold in the direction of movement of the spout or bud. The flaps or tabs may be separately formed and then attached to the body portion of the implant or may, instead, be integrally formed with the body portion. For example, where the flaps are desired to be tissue infiltratable, the flaps may be an extension of a tissue infiltratable portion of the implant. In the embodiment illustrated in FIGS. 1-3, then, the layer of tissue infiltratable fabric does not end at the opening but, rather, includes several flaps that extend into the opening and are moveable out of the way of the opening upon passage of the bowel during externalization. Where non-tissue infiltratable flaps are desired, then such flaps may be an extension of the barrier portion. It also is contemplated that the flaps may have tissue infiltration and/or barrier properties different than the body portion. For example, the body portion may be tissue infiltatable while the flaps constitute a barrier to tissue ingrowth.

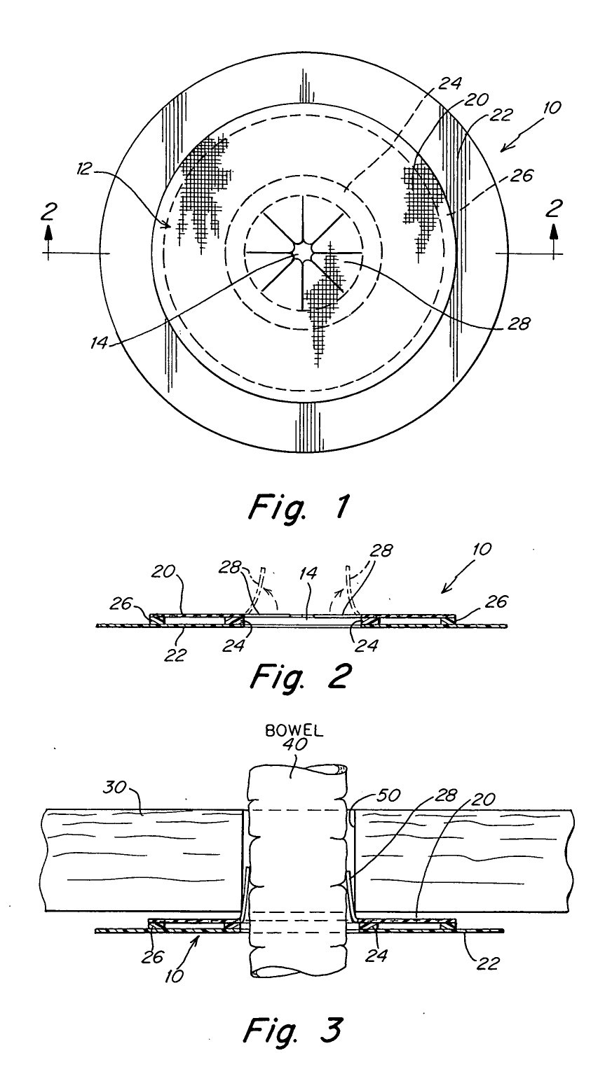

For the purposes of this patent specification, as well as any claims related thereto, the feature of an “opening” adapted to receive the bowel or other anatomical structure shall include a complete opening that is configured to completely surround the bowel, and a partial opening that is configured to only partially surround the bowel, even though the qualifier of “complete” or “partial” is not used. In certain embodiments, the opening in the implantable prosthesis is a non-interrupted complete opening, and the body portion 12 does not include a slit extending between the opening 14 and an edge of the implantable prosthesis. The opening may have a round shape or any other shape that is constructed and arranged to receive the structure that will protrude through the stoma. Where the implantable prosthesis includes tissue infiltratable and barrier portions, the respective portions may have essentially the same size and/or shape, or alternatively may have different sizes and/or shapes. For example, and without limiting the scope of the invention, as shown in FIGS. 2-3, the first layer 20 may extend to the shape influencing member, while the second layer 22 may extend beyond the shape influencing member.

As mentioned above, the present invention includes a method of externalizing a section of bowel or other anatomical structure through an implantable prosthesis, but without contacting the prosthetic device, avoiding contamination thereof and potential infection. This procedure will now be described in connection with an enterostomy. However, the surgical technique is not so limited, and may be employed in the relocation of other anatomical structures, as should be apparent to one of skill in the art.

As illustrated in FIG. 4, a shield, such as a cannula 16, may be provided to allow passage of the structure to be externalized without contact and/or transmission of bacteria or other undesirable to the implantable prosthesis during externalization. The cannula may be translucent to aid visibility of any contents, and preferably remains patent or open without the need of external support. The cannula 16 has an outer dimension 106 that is sized to fit within the opening 14 in the implantable prosthesis 10, and an inner dimension sized to pass an element to be externalized, such as a resected end of bowel 40. The cannula has a length sufficient to span the thickness of the abdominal wall, and preferably any overlying fat and skin, and to project slightly into the abdominal cavity, as explained in more detail below. An opening is formed through the abdominal wall that is adapted to receive the cannula. Preferably, the opening is formed by a trocar stylet 18, such as shown in FIG. 4. The trocar stylet 18 may have an outer dimension 104 sized to fit within the cannula 16, and a sharpened end 42 adapted to puncture through skin, subcutaneous fat, and the abdominal wall. As explained in further detail below, a sizer 44 may also be included to measure the bowel 40 prior to any surgical repair, so that the smallest sized trocar stylet 18 and cannula 16 can be selected. As shown, the trocar stylet 18 slidably fits within the cannula 16 to form a trocar assembly 46. In the illustrated embodiment of FIG. 4, the outer dimension 104 of the cutting tool or stylet 16 is less than the outer dimension 106 of the cannula 106. Accordingly, the opening 50 formed in the abdominal wall 30 is slightly smaller than the outer dimension 106 of the cannula 16. In this respect, the cannula 16 may cause the opening to stretch.

As illustrated in FIG. 5, the selected sized stylet 18 and/or cannula 16 is penetrated through the anatomical wall, creating the stoma. When forming a stoma in the abdominal wall 30, the cannula and stylet assembly typically passes through skin 82, subcutaneous tissues 84, anterior and posterior rectus sheath, 86, 90, rectus muscle 88, and finally through the peritoneum 92 before reaching the abdominal cavity. In an embodiment related to the formation of a stoma in the abdomen, a small incision may be made in the anterior rectus sheath and some of the fibers of the rectus muscle are divided or retracted to clear an avascular path for the cannula 16. In some instances, the inferior epigastric vessels are ligated and divided. In certain embodiments, a stylet is not used; rather other cutting instruments are employed to form or initiate an opening through the anatomical wall through which the cannula is then placed.

With the cannula in place, and the stylet or other cutting tools removed, the opening of the implantable prosthesis 10 is ready to be registered with the entrance to the stoma formed in the abdominal wall. The implantable prosthesis 10 may be inserted into the abdominal cavity either by an open or laparoscopic approach. For example, the prosthesis 10 may be inserted through the opening 50 in the anatomical structure created by the insertion of the cannula 16, either through a vacant stoma or down the lumen of the cannula if the cannula is in place. Alternatively, the prosthesis may be inserted through another opening into the abdominal cavity, such as a second opening somewhat adjacent to the stoma. Regardless of the method of insertion into the anatomical structure, the opening 14 of the prosthesis 10 may now be located about the protruding end of the cannula 16, in registry with the entrance to the stoma, and with the body portion adjacent the abdominal wall surrounding the stoma. In certain embodiments, the prosthesis 10 may be rearwardly spaced from the end 48 of the cannula 16. The distance between the prosthetic patch and the end of the cannula may range from a millimeter to as much as several centimeters, or more. The spacing between the end of the cannula and the implantable prosthesis being selected to reduce the incidence of contact, and/or reduce the likelihood of transmission of bacteria or other undesirables, between the bowel and the implantable prosthesis when the sectioned end of bowel is externalized.

As previously stated, it may be particularly desirable to avoid contact between the tip 54 of the bowel 40 and the implantable prosthesis 10, since the tip 54 may be contaminated by bacteria or other undesirables. Typically, the bowel 40 is resected prior to this procedure, therefore the tip 54 of the bowel is temporarily sealed, for example with staples 56. Since the staples 56 may not provide a complete seal, it may be desirable to limit, if not avoid any, contact between the tip 54 of the bowel 40 and the implantable prosthesis 10 to lessen the risk of infection and other resulting complications.

With the opening 14 of the implantable prosthesis 10 positioned about the end of the cannula 16, the prosthesis may be attached to the abdominal wall 30, for example, with sutures 58, 60. The sutures 58, 60 may be non-absorbable, and they may be placed intermittently or continuously about the prosthesis 10. In one embodiment, the sutures extend about the periphery of the implantable prosthesis 10, into the parietal peritoneum, the underlying posterior rectus sheath and deeply into the fascia. Other arrangements for joining the implantable prosthesis to the abdominal wall may be employed as should be apparent to one of skill in the art. On the other hand, as observed earlier, no fixation devices are required and the implantable prosthesis may be held in place by engagement with the cannula, attraction to the abdominal wall, body forces and/or otherwise.

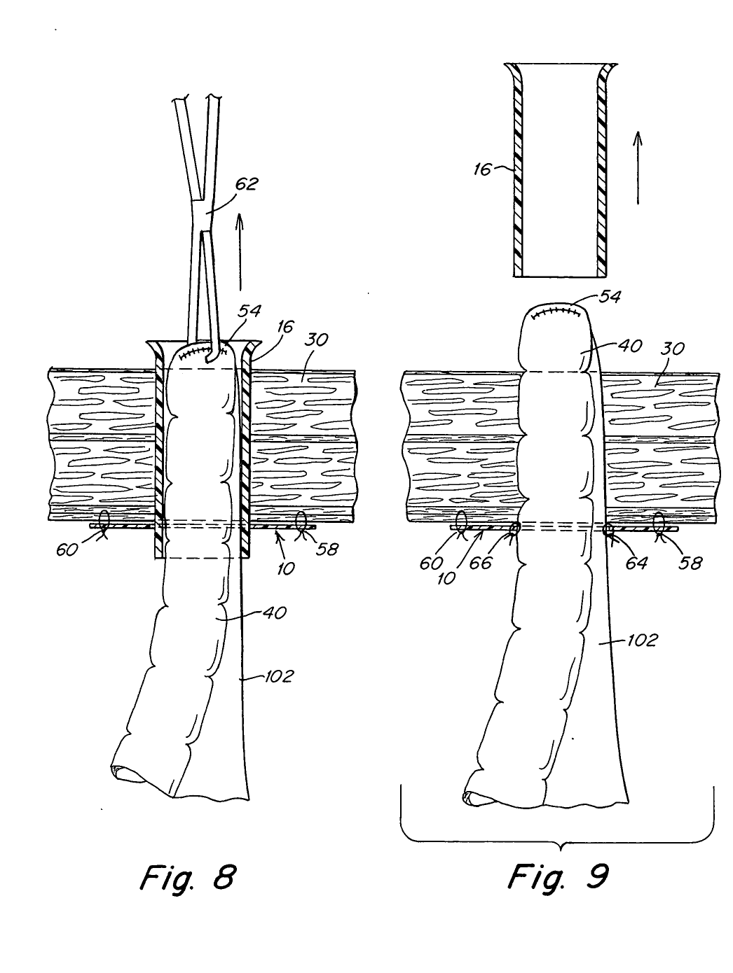

With the implantable prosthesis 10 in place, a portion of the bowel 40 may be inserted into the end 48 of the cannula 16 that protrudes into the abdominal cavity, and may be advanced through the cannula to the outside. Due to the position of the implantable prosthesis rearward of the end of the cannula 16 protruding into the abdominal cavity, the bowel 40 should not come in contact with the prosthesis during externalization, and thus limits the potential for bacterial contamination of the implant. Further, the abdominal wall and overlying tissue which has been exposed during formation of the stoma is isolated by the cannula from the potentially unsterile intestine as it is relocated through the abdominal wall to outside of the patient. In one embodiment, a grasping instrument 62 (FIG. 7) may be inserted through the cannula 16 and into the abdominal cavity to grab and draw the bowel 40 out of the abdominal cavity (FIG. 8). It may be preferable to draw the tip 54 of the bowel 40 up past the skin surface by about 1-2 inches to provide sufficient room to form the stoma bud 108. The bowel 40 may also be passed through the cannula 16 using other instruments and methods as should be apparent to one of skill in the art.

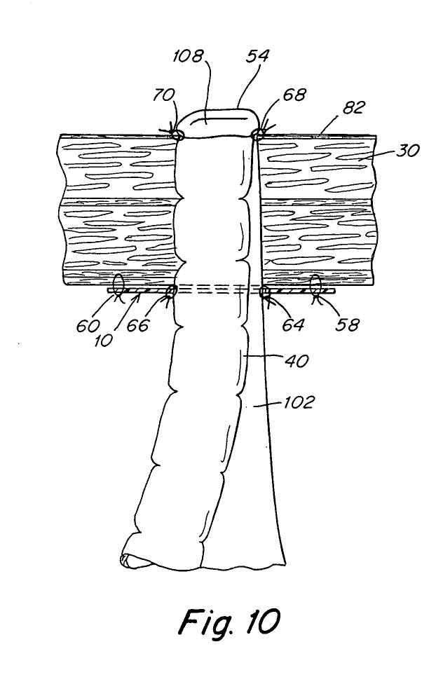

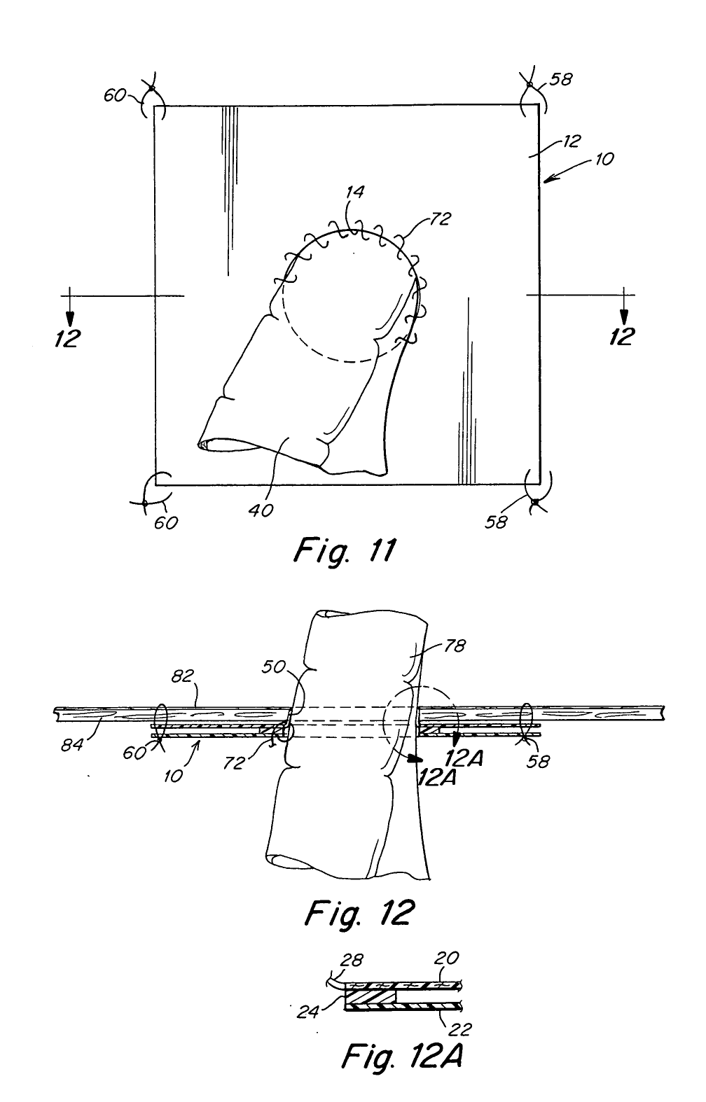

As illustrated in FIG. 9, once the bowel 40 is brought to the skin surface, the cannula 16 may be removed. It may be desirable, then, to secure the implantable prosthesis 10 to the bowel 40 and its mesentery 102. For example, sutures, 64, 66 may be positioned about the opening 14 of the prosthesis and the outer surface of the bowel 40 and mesentery 102. Other arrangements for fixing the implant to the bowel may be employed as should be apparent to one of skill in the art. In one embodiment, a purse-string continuous suture 72 may be used to secure the implantable prosthesis 10 to the bowel 40 as shown in FIG. 11. It is not, however, critical that the bowel be joined to the implantable prosthesis. As illustrated in FIG. 12, the implantable prosthesis 10 may be located intra-peritoneally. Other placements of the patch, such as pre-peritoneal, also are contemplated. As shown in FIG. 10, the spout or bud may further be secured to the skin 82, for example with sutures 68, 70.

As illustrated in FIG. 12, the implantable prosthesis may be reinforced at or near the opening to reduce the incidence of herniation at the stoma. In the embodiment illustrated, a reinforcement member 24 is positioned between a first tissue infiltratable layer 20 and a second barrier layer 22. Further, as previously described, flaps 28 may extend into the opening 14 of the implantable prosthesis to further promote, or discourage, the adherence between the prosthesis 10 and the element 78 which extends therethrough.

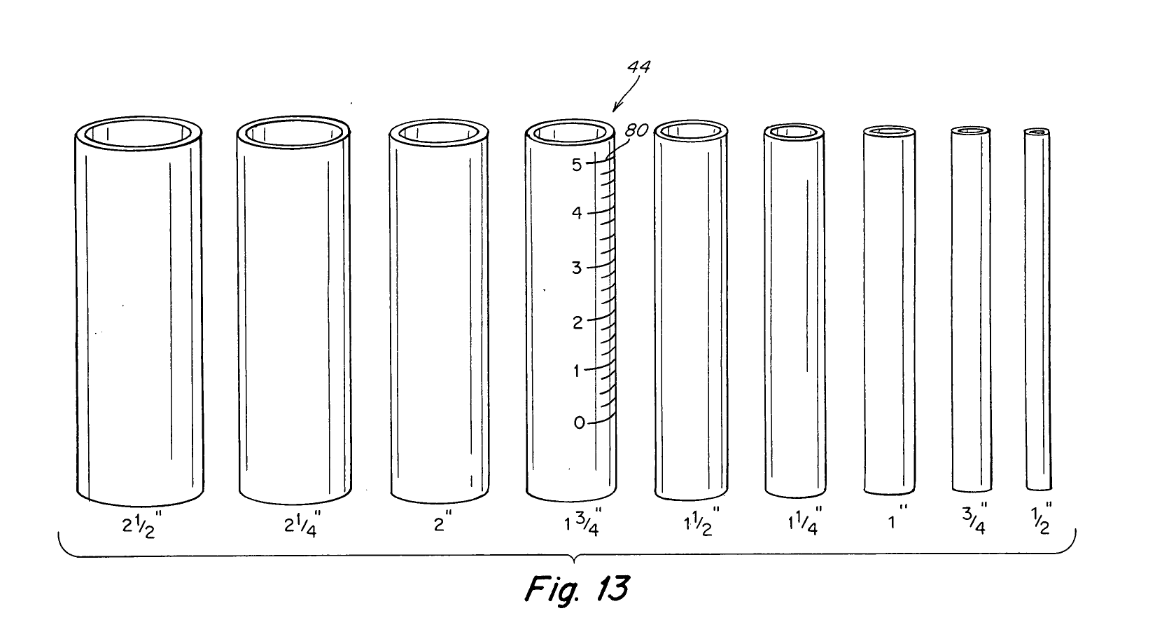

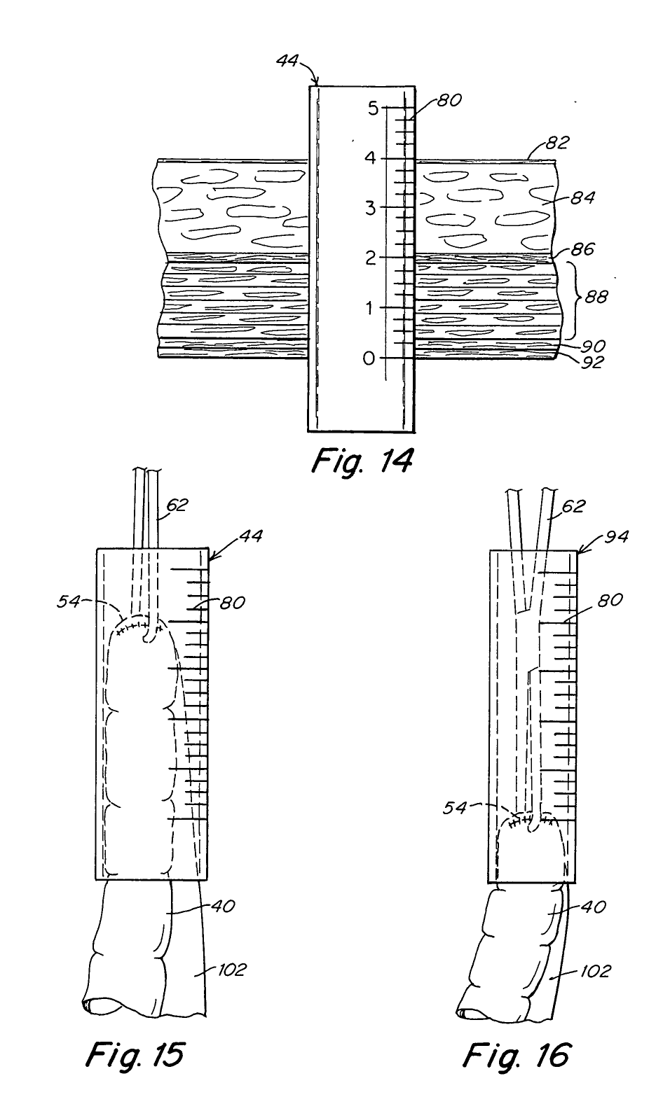

It may be desirable to match the opening that will be formed by the trocar stylet to the thickness or width of the externalized section of intestine that will extend through the abdominal wall. Accordingly, the depth of the abdominal wall may be measured, as shown in FIG. 14, and to that measurement is added the length of spout or bud that is expected to protrude from the skin surface of the patient. A length of intestine equal to the thickness of the abdominal wall and the protruding segment, may be inserted into a series of sizers until a sizer is found having an inner dimension that matches the thickness at the end of the intestine length (See FIG. 15). That sizer has a corresponding trocar stylet which will form a passageway through the abdominal wall of the selected size to approximate the thickness of the end of the intestine. Each sizer 44 may be constructed from a sterilizable material, such as a translucent plastic, and may further include a measurement scale 80. The measurement scale 80 may be offset from one end of the sizer 44 by a predetermined amount, for example by approximately 1.5 inches, to correspond to the distance that the bowel should protrude from the skin surface prior to maturing the stoma bud 108.

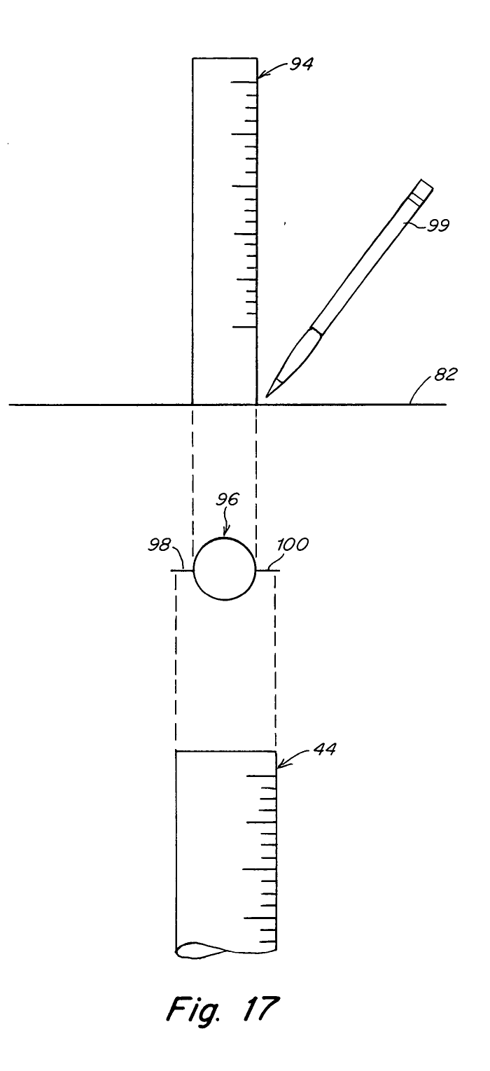

Further, as illustrated in FIGS. 16-17, the sizer 94 that will just admit the tip 54 of the bowel 40 may be used as a template to mark the skin 82 for forming the buttonhole 96 for the stomal bud 108, as further described below. The outer dimension of the buttonhole 96 preferably corresponds to the outer dimension of the sizer 94. The size of the buttonhole 96 may be smaller than the size of the cannula 16 selected to pass through the abdominal wall 30, particularly in the case of obese patients having a thick mesentery 102. To allow advancement of the cannula 16 through this smaller opening, small lateral and medial incisions may be made to temporarily enlarge the buttonhole 96. The length of the lateral and medial incisions 98, 100 extending out from the buttonhole 96 may extend out to the outer dimension of the sizer 44. The lateral and medial incisions 98, 100 may be necessary to temporarily enlarge the opening in the abdominal wall 30 to accommodate the cannula 16. If these incisions are employed, they may be subsequently closed with fine absorbable sutures to reduce the size of the incision in the abdominal wall to its original size.

This above described measurement system with the sizers 94 and/or 44 provide an ability to customize the method of creating a stoma to the individual patient. This sizing method further limits the opening created in the abdominal wall to only as large as desired for the passing of a portion of the bowel 40 up to the skin surface. Accordingly, the stoma created preferably approximates the thickness of the externalized bowel section. This in itself may help to prevent a herniation of the surrounding areas.

The order of steps of the inventive method described above are not critical to the invention. For example, and without limiting the foregoing, the implantable prosthesis may be positioned against the abdominal wall and then the cannula end inserted therethrough, or instead the cannula may be inserted through the abdominal wall and then the implantable prosthesis mounted about the cannula end protruding into the abdominal cavity. Further, the bowel section may be drawn into the cannula before or after the cannula and implantable prosthesis have been combined together; although it would seem preferable to insert the bowel section into the cannula after the cannula has been positioned relative to the implantable prosthesis to reduce the likelihood of contact, and/or bacteria transmission, between the bowel and the implant.

A kit may be provided including an implantable prosthesis and a cannula for externalizing a bowel portion or other anatomical or prosthetic structure as previously described herein. The kit may also include one or more sizing cylinders and one or more trocar stylets, as well as additional cannulas that correspond to the various trocar stylets. Still further, the kit or a system including the implantable prosthesis and cannula, and sizing cylinders and/or trocar stylets if so provided, may include instructions for using any such component in the repair, or resistance to formation, of a stoma hernia, and/or in the externalization of an anatomical or prosthetic structure through a stoma.

It should be understood that the foregoing description of the invention is intended merely to be illustrative thereof and that other equivalents, embodiments and modifications of the invention may be apparent to those skilled in the art.

1. A system for repairing, or resisting the formation of, a hernia at or near a stoma formed in an abdominal wall, wherein a portion of bowel extends through the stoma, said system comprising:
an implantable prosthesis having a body portion and an opening therethrough that is adapted to receive the portion of the bowel, said body portion being effective for at least one of the repair, or resistance to formation, of an abdominal wall hernia at or near the stoma; and
a cannula, separate from said implantable prosthesis, having an outer dimension that is sized to fit within the opening in said implantable prosthesis, and an inner dimension that is sized to pass the portion of bowel therethrough, said cannula being removable from said opening in said implantable prosthesis.

2. The system of claim 1 wherein said body portion includes at least one of a tissue infiltratable portion and a barrier portion.

3. The system of claim 2, wherein said body portion includes a first layer that is tissue infiltratable and a second layer that is a barrier.

4. The system of claim 3 wherein said first layer is a top surface, and said second layer is a bottom surface.

5. The system of claim 4 wherein said first layer is a porous fabric and said second layer is ePTFE.

6. The system of claim 1 wherein a portion of said implantable prosthesis is reinforced at or near said opening to resist enlargement of said opening, said reinforced portion having at least one property of strength, resistance to elongation and stiffness, that is superior to an adjacent aspect of said body portion.

7. The system of claim 6 wherein said implantable prosthesis includes a separate reinforcement member connected to a surface of said body portion at or near said opening.

8. The system of claim 7 wherein said reinforcement member is a ring.

9. The system of claim 7 wherein said reinforcement member extends at least partially around said opening.

10. The system of claim 7 wherein said body portion includes a first tissue infiltratable layer and a second barrier layer, and said implantable prosthesis is reinforced between said first and second layers.

11. The system of claim 7 wherein said body portion does not include a slit extending between said opening and an edge of said implantable prosthesis.

12. The system of claim 1 further including instructions for externalizing the bowel section through said cannula, limiting the potential for contact and/or transmission of bacteria or other undesirables between the bowel section and said implantable prosthesis.

13. The system of claim 1 including a kit provided with an implantable prosthesis and a cannula.

14. The system of claim 1, further comprising a trocar stylet having an outer dimension that is sized to fit within said cannula, and a first end adapted to puncture the anatomical wall.

15. The system of claim 1 further including at least one flap extending across said opening.

16. The system of claim 15 wherein said at least one flap is at least one of said tissue infiltratable portion and said barrier portion.

17. The system of claim 16 wherein said at least one flap includes a plurality of flaps that extend about said opening.

18. The system of claim 1 further including at least one sizer for determining the size of a trocar stylet for forming the stoma.

19. The system of claim 1 further including at least one shape influencing member.

20. A method of repairing, or reducing the incidence of formation of, a hernia at or near a stoma formed in an abdominal wall for externalizing a portion of bowel, the method comprising the steps of:
providing an implantable prosthesis with an opening therethrough that is adapted to receive the bowel portion;
providing a shield having an outer dimension that is sized to fit within the opening of said implantable prosthesis and an inner dimension that is sized to pass the bowel portion therethrough;
inserting the shield into and through the stoma;
positioning at least one of the shield and the implantable prosthesis, so that an end of the shield extends through the opening in the implantable prosthesis;
inserting the bowel portion into the shield; and
removing the shield.

21. The method of claim 20, wherein the shield is a cannula.

22. The method of claim 21 further comprising the steps of:
providing a trocar stylet having an outer dimension that is sized to fit within the cannula, and a first end adapted to puncture the abdominal wall to form the stoma; and
inserting said trocar stylet with the cannula into the abdominal wall.

23. The method of claim 21, further comprising the step of:
positioning the implantable prosthesis rearward of the end of the cannula that has been passed through the stoma and into the abdominal cavity.

24. The method of claim 23, wherein the prosthesis is spaced from the cavity entering end of the cannula by at least 1 cm.

25. The method of claim 21 wherein the cannula is extended through the opening before said step of inserting said bowel portion into the cannula.

26. The method of claim 22, further comprising the step of:
sizing the bowel section with a sizing cylinder to determine an appropriately sized trocar stylet for forming the stoma.

27. The method of claim 20 including at least one of a colostomy, ileostomy and a urostomy.
